(19) United States
(12) Patent Application Publication
(10) Pub. No.: US 2011/0218434 A1
(43) Pub. Date: Sep. 8, 2011

(54) Workflow Driven User Interface for a Power Injection Systems

(76) Inventors: Robert J. Ziemba, Cincinnati, OH (US); Charles S. Neer, Cincinnati, OH (US)

(21) Appl. No.: 13/000,463

(22) PCT Filed: Nov. 25, 2009
(86) PCT No.: PCT/US09/65857

§ 371 Date: Dec. 21, 2010

Related U.S. Application Data
(60) Provisional application No. 61/118,515, filed on Nov. 28, 2008.

(51) Int. Cl. A61B 6/00

(52) U.S. Cl. 600/432

(57) ABSTRACT

A medical fluid delivery system (300) includes workflow construction logic (304) to create one or more workflows (307) from a plurality of available workflow components (306). The created workflows (307) may each contain a proper subset of workflow components from the plurality of available workflow components (306). The workflows (307) may be created using the workflow construction logic (304) and a user interface (302) by selecting and arranging graphical elements representing workflow components (306). The constructed workflows (307) may correspond with the practices of a given care group. The medical fluid delivery system (300) may further include workflow execution logic (305) operable to execute the workflows (307). When executed, the workflows (307) may sequentially guide a user through each of the included workflow components (306) using a fled interface. The nomenclature displayed by the workflow components (306) during workflow execution may be customizable.

### CROSS-REFERENCE TO RELATED APPLICATIONS

This patent application claims priority under 35 U.S.C. §119(e) to pending U.S. Patent Provisional Patent Application Ser. No. 61/118,515 entitled “Workflow Driven User Interface For A Power Injection System” filed on 28 Nov. 2008, the entire disclosure of which is hereby incorporated by reference herein to the extent not inconsistent with this disclosure.

### FIELD OF THE INVENTION

The present invention generally relates to the field of medical fluid delivery systems and, more particularly, to the field of user interfaces for setup and operation of medical fluid delivery systems.

### BACKGROUND

Various medical procedures require that one or more medical fluids be injected into a patient. For example, medical imaging procedures oftentimes involve the injection of contrast media into a patient, possibly along with saline and/or other fluids. Other medical procedures involve injecting one or more fluids into a patient for therapeutic purposes. Power injectors may be used for these types of applications.

A power injector generally includes what is commonly referred to as a powerhead. One or more syringes may be mounted to the powerhead in various manners (e.g., detachably; rear-loading; front-loading; side-loading). Each syringe typically includes what may be characterized as a syringe plunger, piston, or the like. Each such syringe plunger is designed to interface with (e.g., contact and/or temporarily interconnect with) an appropriate syringe plunger driver that is incorporated into the powerhead, such that operation of the syringe plunger driver axially advances the associated syringe plunger inside and relative to a barrel of the syringe. One typical syringe plunger driver is in the form of a ram that is mounted on a threaded lead or drive screw. Rotation of the drive screw in one rotational direction advances the associated ram in one axial direction, while rotation of the drive screw in the opposite rotational direction advances the associated ram in the opposite axial direction.

The user interfaces on power injectors are generally organized such that at any given time, a variety of capabilities of the power injector may be accessed. This flexibility typically requires a level of complexity to the user interface. Generally, users rely on training to know the sequence of steps required by their organization to inject medical fluids. Typically, at each step, the user goes through a menu, locates the step to be performed, and initiates the step.

### SUMMARY

The present invention is generally directed to the creation of workflows in the context of medical applications, specifically in the context of the delivery of medical fluids. Generally, a workflow is a set of workflow components arranged in a predetermined order such that when the workflow is executed, the workflow components are performed in the predetermined order to perform a procedure using a medical fluid delivery system. Such a procedure may, for example, include setting up the medical fluid delivery system and then injecting a patient with medical fluid (e.g., contrast media and/or saline) with the medical fluid delivery system. A workflow component is a discrete task or set of tasks that may be performed in connection with the use of the medical fluid delivery system. For example, a workflow component may include instructions to load or mount a prefilled syringe onto the medical fluid delivery system.

A first aspect of the present invention is embodied by a medical fluid delivery system that includes an injection device, a user interface, and workflow construction logic. The workflow construction logic is operable to present a plurality of available workflow components to a user through the user interface. The workflow construction logic is operable to receive a selection of a proper subset of the plurality of available workflow components from the user through the user interface. The workflow construction logic is operable to construct a workflow from the proper subset.

A number of feature refinements and additional features are applicable to the first aspect of the present invention. These feature refinements and additional features may be used individually or in any combination. As such, each of the following features that will be discussed may be, but are not required to be, used with any other feature or combination of features of the first aspect. The following discussion is applicable to the first aspect, up to the start of the discussion of a second aspect of the present invention.

The user interface may be integral with the injection device (e.g., incorporated by a display of a power injector). The injection device may include the workflow construction logic. For instance, the workflow construction logic may be integrated into a powerhead of a power injector. In an arrangement, the user interface, the injection device, and the workflow construction logic may be a unitary device.

The plurality of available workflow components may include mutually exclusive workflow components. At least some of the workflow components that are available for selection may be mutually exclusive. The workflow construction logic may be operable to prevent the simultaneous selection of mutually exclusive components during construction of a workflow. Similarly, the workflow construction logic may be operable to prevent the simultaneous inclusion of mutually exclusive components in the workflow. The plurality of available workflow components may include workflow components related to one or more of the following: syringe type selection, syringe quantity selection, injection protocol selection, syringe loading, syringe re-loading, syringe filling, tubing set connecting and purging, patency check, test injection, main injection, results display, results printing, and tubing and syringe removal.

The workflow construction logic may allow for alteration of visual elements displayed on the user interface in association with at least a portion of the plurality of available workflow components. Such alterations may take the form of changing textual elements associated with the visual elements. In this regard, displays related to particular workflows may contain customized textual descriptions and/or labels.

The medical fluid delivery system may further include workflow execution logic operable to present the workflow that has been constructed to a user. The workflow execution logic may initiate functionality of the medical fluid delivery system. The workflow execution logic may operate to sequentially execute each workflow component of the proper subset of the plurality of available workflow components in a predetermined sequence. The medical fluid delivery system may include a plurality of stored workflows. The medical fluid delivery system may include a data input device that allows for selection of a workflow from the plurality of stored workflows. The workflow execution logic may generate at least one output on a display for each workflow component of the workflow. The workflow execution logic may generate a sequence of outputs on a display in accordance with the workflow.

The workflow execution logic may include a suspension function that operates to temporarily suspend performance of the workflow. While the workflow is temporarily suspended, the medical fluid delivery system may be operable to perform functions that are not part of the workflow. The workflow execution logic may be operable to resume the workflow after completion of the suspension function.

The second aspect of the present invention is embodied by a method of operating a medical fluid delivery system. The method includes providing a medical fluid delivery system that includes a plurality of available workflow components, and selecting a proper subset of workflow components from the plurality of available workflow components for inclusion within a workflow.

A number of feature refinements and additional features are applicable to the second aspect of the present invention. These feature refinements and additional features may be used individually or in any combination. As such, each of the following features that will be discussed may be, but are not required to be, used with any other feature or combination of features of the second aspect. The following discussion is applicable to the second aspect, up to the start of the discussion of a third aspect of the present invention.

The method of operating the medical fluid delivery system of the current aspect may further include arranging workflow components of the proper subset to form the workflow. The arranging may be performed by a user (e.g., an administrator), by the medical fluid delivery system (e.g., initiated by workflow construction logic), or by a combination of the user and the medical fluid delivery system. Where at least a portion of the arranging is performed by a user, the arranging may, for example, include any appropriate method of moving components on a display, such as dragging and dropping a graphical element on the display that represents the workflow component. The medical fluid delivery system may indicate to a user that a workflow component is in an inappropriate position relative to another workflow component within the workflow being created. The medical fluid delivery system may prevent (e.g., during the selecting and/or arranging steps) inclusion of workflow components within the workflow that are mutually exclusive. The arranging of workflow components may be accompanied by visually displaying visual elements (e.g., icons and/or blocks of text) that represent each of the workflow components. Visual elements representing mutually exclusive workflow components may be displayed with a common color, a common pattern, a common shape and/or any other appropriate trait (or combination of traits) to indicate their relationship.

A plurality of visual elements may be displayed on a user interface. Each of the plurality of visual elements may represent at least one workflow component of the plurality of available workflow components. The selection of workflow components for a workflow may include moving one of the visual elements from a first portion of the user interface to a second portion of the user interface. This may be achieved in any appropriate way, such as by selecting the visual element and having the medical fluid delivery system move the element to the second portion of the user interface, or by dragging and dropping the element to the second portion of the user interface. The first portion of the user interface may include a plurality of visual elements representing unselected workflow components and the second portion of the user interface may include a plurality of visual elements representing selected workflow components.

In an arrangement, the selection of workflow components for a workflow may comprise including within the workflow a workflow component that includes an adjustable parameter. The method may include inputting a default value and/or a default range for the adjustable parameter during the selection of workflow components for a workflow and/or the arranging of selected workflow components for the workflow. After the workflow components have been selected and arranged, a user may input (e.g., as part of an injection or discharge of medical fluid from the medical fluid delivery system) a value for the adjustable parameter.

The method may further include displaying a representation of a partially constructed as the workflow components are being selected and/or arranged. The method may further include customizing textual elements of a display. The textual elements may be textual elements associated with workflow components, such as the workflow components associated with the proper subset and/or the plurality of available workflow components. The textual elements may be associated with instructions displayed during execution of a workflow component. The method may further include providing a password to the medical fluid delivery system to enable generation of a workflow. The workflow may be password protected such that the likelihood of unauthorized and/or unintentional creation, alteration, and/or deletion of workflows may be reduced.

The method may include performing at least a portion of the workflow to prepare the medical fluid delivery system for injecting medical fluid into a patient. In this regard, performing the workflow may initiate functionality of the medical fluid delivery system. A portion of the workflow may include workflow components related to mounting a syringe onto the medical fluid delivery system. A portion of the workflow may include workflow components related to purging portions of the medical fluid delivery system of air.

The performed portion of the workflow may include displaying instructions for a user (e.g., clinician or medical technician) and/or automatic performance of at least a portion of a workflow component of the proper subset by the medical fluid delivery system. The performance of the workflow may include sequentially displaying a first display (e.g., a first output on a display) associated with a first workflow component and a second display (e.g., a second output on a display) associated with a second workflow component. The first display may be configured such that it does not display any information related to the second workflow component. The second display may be configured such that it does not display any information related to the first workflow component. In this regard, the first and second displays may be simplified to clearly communicate information to a user related to the current workflow component. The method may further include discharging medical fluid from the medical fluid delivery system.

The method of operating the medical fluid delivery system may include executing the workflow to inject medical fluid into a patient. The execution of the workflow may include first selecting a protocol and then the medical fluid delivery system may select the workflow based on the protocol that has been selected. Alternatively, the execution of the workflow may include first selecting and executing the workflow, and selection of an injection protocol may be a component of the workflow. The selection of the proper subset of workflow components may, for example, be performed by an administrator, while the execution of the workflow may be performed by a clinician or medical technician.

The selection of the proper subset of workflow components may include assigning a default value related to a workflow component of the proper subset. Such an assignment may be performed by a user of the medical fluid delivery system. Subsequently, the execution of the workflow may be performed using the default value. A user of the medical fluid delivery system may override the default value while executing the workflow.

The selection of the proper subset of workflow components may include assigning a default range of values related to a workflow component of the proper subset. Such an assignment may be performed by a user of the medical fluid delivery system. Subsequently, the execution of the workflow may be performed using the default range. A user of the medical fluid delivery system may override the default range and perform the workflow component with a value outside of the default range while executing the workflow.

The execution of the workflow may include sequentially performing each workflow component of the proper subset of workflow components. The execution of the workflow may include presenting instructions for a user (e.g., clinician or medical technician) on a first display and/or automatic performance of at least a portion of a workflow component of the proper subset of workflow components by the medical fluid delivery system.

The execution of the created a workflow may include sequentially presenting a first output on a first display associated with a first workflow component and then presenting a second output on the first display associated with a second workflow component. The presenting of the second output associated with the second workflow component may occur automatically after completion of the first workflow component.

The method may further include exiting the workflow prior to completion of each workflow component, and performing a task with the medical fluid delivery system that deviates from the workflow. The method may further include resuming the workflow at the point where it was exited. For instance, the execution of a workflow may be suspended for one or more reasons, and thereafter may be re-initiated.

The method of operating the medical fluid delivery system may further include saving the workflow after the workflow components have been selected. The method may further include selecting the workflow after the workflow has been saved and before being executed. For example, an administrator may select steps to be included into the workflow and save the workflow. Subsequently, a clinician may execute the saved workflow. The selection of workflow components may include choosing the workflow from a plurality of stored workflows. Such a selection may be performed with a data input device of any appropriate type. A user (e.g., an administrator) may designate the workflow as a default workflow after the workflow has been saved and before execution of the workflow has been initiated.

The third aspect of the present invention is provided by a medical fluid delivery system that includes an injection device and a memory unit. The memory unit includes at least one injection protocol containing injection execution parameters, a plurality of workflow components, and a first workflow. The first workflow includes a first proper subset of the plurality of workflow components. The first workflow does not include any workflow components not contained in the first proper subset.

A number of feature refinements and additional features are applicable to the third aspect of the present invention. These feature refinements and additional features may be used individually or in any combination. As such, each of the following features that will be discussed may be, but are not required to be, used with any other feature or combination of features of the third aspect. The following paragraph is applicable to the third aspect.

The medical fluid delivery system may further include a plurality of injection protocols each containing injection execution parameters. The medical fluid delivery system may further include a second workflow that includes a second proper subset of the plurality of workflow components. The second workflow may not include any workflow components not contained in the second proper subset. The first proper subset may be different than the second proper subset.

As used herein, the expression “operable to” indicates a relationship where an item is capable of performing the specified task and/or achieving the specified result. As used herein, the meaning of “operable to” is intended to encompass similar expressions such as “adapted to” and “configured to.” Furthermore, as used herein, where an item is “operable to” perform a function, it is also disclosed that the item does perform that function in a least some situations. Therefore, wherever “operable to” is used, relationships between the item and the specified task and/or achievement of the specified result are disclosed. Accordingly, where an item is “operable to” perform a task, it is also disclosed that the item performs that task (e.g., the item “operates to” perform the task).

As used herein, the term “fluidly interconnected” refers to two or more components or entities being connected (directly or indirectly) in a manner such that fluid can flow (e.g., unidirectionally or bidirectionally) in a predetermined flow path therebetween. For example, “an injection device fluidly interconnected to a patient” describes a configuration where fluid can flow from the injection device through any interconnecting devices (e.g., tubing, connectors) and into the patient (e.g., into the vasculature of the patient).

A number of feature refinements and additional features are separately applicable to each of above-noted first, second, and third aspects of the present invention. These feature refinements and additional features may be used individually or in any combination in relation to each of the above-noted first, second, and third aspects. Any feature of any other various aspects of the present invention that is intended to be limited to a “singular” context or the like will be clearly set forth herein by terms such as “only,” “single,” “limited to,” or the like. Merely introducing a feature in accordance with commonly accepted antecedent basis practice does not limit the corresponding feature to the singular (e.g., indicating that a power injector includes “a syringe” alone does not mean that the power injector includes only a single syringe), Moreover, any failure to use phrases such as “at least one” also does not limit the corresponding feature to the singular (e.g., indicating that a power injector includes “a syringe” alone does not mean that the power injector includes only a single syringe). Finally, use of the phrase “at least generally” or the like in relation to a particular feature encompasses the corresponding characteristic and insubstantial variations thereof (e.g., indicating that a syringe barrel is at least generally cylindrical encompasses the syringe barrel being cylindrical).

Any “logic” that may be utilized by any of the various aspects of the present invention may be implemented in any appropriate manner, including without limitation in any appropriate software, firmware, or hardware, using one or more platforms, using one or more processors, using memory of any appropriate type, using any single computer of any appropriate type or a multiple computers of any appropriate type and interconnected in any appropriate manner, or any combination thereof. This logic may be implemented at any single location or at multiple locations that are interconnected in any appropriate manner (e.g., via any type of network).

Any power injector that may be utilized to provide a fluid discharge may be of any appropriate size, shape, configuration, and/or type. Any such power injector may utilize one or more syringe plunger drivers of any appropriate size, shape, configuration, and/or type, where each such syringe plunger driver is capable of at least bi-directional movement (e.g., a movement in a first direction for discharging fluid; a movement in a second direction for accommodating a loading and/or drawing of fluid and/or so as to return to a position for a subsequent fluid discharge operation), and where each such syringe plunger driver may interact with its corresponding syringe plunger in any appropriate manner (e.g., by mechanical contact; by an appropriate coupling (mechanical or otherwise)) so as to be able to advance the syringe plunger in at least one direction (e.g., to discharge fluid). Each syringe plunger driver may utilize one or more drive sources of any appropriate size, shape, configuration, and/or type. Multiple drive source outputs may be combined in any appropriate manner to advance a single syringe plunger at a given time. One or more drive sources may be dedicated to a single syringe plunger driver, one or more drive sources may be associated with multiple syringe plunger drivers (e.g., incorporating a transmission of sorts to change the output from one syringe plunger to another syringe plunger), or a combination thereof. Representative drive source forms include a brushed or brushless electric motor, a hydraulic motor, a pneumatic motor, a piezoelectric motor, or a stepper motor.

Any such power injector may be used for any appropriate application where the delivery of one or more medical fluids is desired, including without limitation any appropriate medical application (e.g., computed tomography or CT imaging; magnetic resonance imaging or MRI; single photon emission computed tomography or SPECT imaging; positron emission tomography or PET imaging; X-ray imaging; angiographic imaging; optical imaging; ultrasound imaging). Any such power injector may be used in conjunction with any component or combination of components, such as an appropriate imaging system (e.g., a CT scanner). For instance, information could be conveyed between any such power injector and one or more other components (e.g., scan delay information, injection start signal, injection rate).

Any appropriate number of syringes may be utilized with any such power injector in any appropriate manner (e.g., detachably; front-loaded; rear-loaded; side-loaded), any appropriate medical fluid may be discharged from a given syringe of any such power injector (e.g., contrast media, a radiopharmaceutical, saline, and any combination thereof), and any appropriate fluid may be discharged from a multiple syringe power injector configuration in any appropriate manner (e.g., sequentially, simultaneously), or any combination thereof. In one embodiment, fluid discharged from a syringe by operation of the power injector is directed into a conduit (e.g., medical tubing set), where this conduit is fluidly interconnected with the syringe in any appropriate manner and directs fluid to a desired location (e.g., to a catheter that is inserted into a patient for injection). Multiple syringes may discharge into a common conduit (e.g., for provision to a single injection site), or one syringe may discharge into one conduit (e.g., for provision to one injection site), while another syringe may discharge into a different conduit (e.g., for provision to a different injection site). In one embodiment, each syringe includes a syringe barrel and a plunger that is disposed within and movable relative to the syringe barrel. This plunger may interface with the power injector's syringe plunger drive assembly such that the syringe plunger drive assembly is able to advance the plunger in at least one direction, and possibly in two different, opposite directions.

### BRIEF DESCRIPTION OF THE FIGURES

FIG. 1 is a schematic of one embodiment of a power injector.

FIG. 2A is a perspective view of one embodiment of a portable stand-mounted, dual-head power injector.

FIG. 2B is an enlarged, partially exploded, perspective view of a powerhead used by the power injector of FIG. 2A.

FIG. 2C is a schematic of one embodiment of a syringe plunger drive assembly used by the power injector of FIG. 2A.

FIG. 3A is a block diagram of one embodiment of a medical fluid delivery system.

FIG. 3B is a block diagram of one embodiment of a medical fluid delivery system.

FIG. 4A is a flow diagram of one embodiment of a method of constructing a workflow.

FIG. 4B is a flow diagram of one embodiment of a method of operating a medical fluid delivery system.

FIG. 5 is an illustration of an exemplary display on a user interface of the medical fluid delivery system of FIG. 3A during construction of a workflow.

FIG. 6 is an illustration of an exemplary display on a user interface of the medical fluid delivery system of FIG. 3A during execution of a workflow.

### DETAILED DESCRIPTION

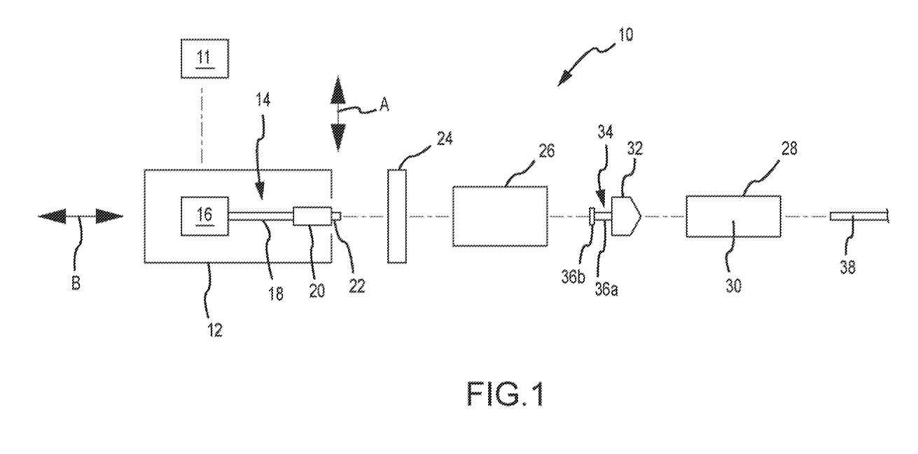

FIG. 1 presents a schematic of one embodiment of a power injector 10 having a powerhead 12. One or more graphical user interfaces or GUIs 11 may be associated with the powerhead 12. Each GUI 11: 1) may be of any appropriate size, shape, configuration, and/or type; 2) may be operatively interconnected with the powerhead 12 in any appropriate manner; 3) may be disposed at any appropriate location; 4) may be configured to provide any of the following functions: controlling one or more aspects of the operation of the power injector 10; inputting/editing one or more parameters associated with the operation of the power injector 10; and displaying appropriate information (e.g., associated with the operation of the power injector 10); or 5) any combination of the foregoing. Any appropriate number of GUIs 11 may be utilized. In one embodiment, the power injector 10 includes a GUI 11 that is incorporated by a console that is separate from but which communicates with the powerhead 12. In another embodiment, the power injector 10 includes a GUI 11 that is part of the powerhead 12. In yet another embodiment, the power injector 10 utilizes one GUI 11 on a separate console that communicates with the powerhead 12, and also utilizes another GUI 11 that is on the powerhead 12. Each GUI 11 could provide the same functionality or set of functionalities, or the GUIs 11 may differ in at least some respect in relation to their respective functionalities.

A syringe 28 may be installed on the powerhead 12 and, when installed, may be considered to be part of the power injector 10. Some injection procedures may result in a relatively high pressure being generated within the syringe 28. In this regard, it may be desirable to dispose the syringe 28 within a pressure jacket 26. The pressure jacket 26 is typically associated with the powerhead 12 in a manner that allows the syringe 28 to be disposed therein as a part of or after installing the syringe 28 on the powerhead 12. The same pressure jacket 26 will typically remain associated with the powerhead 12, as various syringes 28 are positioned within and removed from the pressure jacket 26 for multiple injection procedures. The power injector 10 may eliminate the pressure jacket 26 if the power injector 10 is configured/utilized for low-pressure injections and/or if the syringe(s) 28 to be utilized with the power injector 10 is (are) of sufficient durability to withstand high-pressure injections without the additional support provided by a pressure jacket 26. In any case, fluid discharged from the syringe 28 may be directed into a conduit 38 of any appropriate size, shape, configuration, and/or type, which may be fluidly interconnected with the syringe 28 in any appropriate manner, and which may direct fluid to any appropriate location (e.g., to a patient).

The powerhead 12 includes a syringe plunger drive assembly or syringe plunger driver 14 that interacts (e.g., interfaces) with the syringe 28 (e.g., a plunger 32 thereof) to discharge fluid from the syringe 28. This syringe plunger drive assembly 14 includes a drive source 16 (e.g., a motor of any appropriate size, shape, configuration, and/or type, optional gearing, and the like) that powers a drive output 18 (e.g., a rotatable drive screw). A ram 20 may be advanced along an appropriate path (e.g., axial) by the drive output 18. The ram 20 may include a coupler 22 for interacting or interfacing with a corresponding portion of the syringe 28 in a manner that will be discussed below.

The syringe 28 includes a plunger or piston 32 that is movably disposed within a syringe barrel 30 (e.g., for axial reciprocation along an axis coinciding with the double-headed arrow B). The plunger 32 may include a coupler 34. This syringe plunger coupler 34 may interact or interface with the ram coupler 22 to allow the syringe plunger drive assembly 14 to retract the syringe plunger 32 within the syringe barrel 30. The syringe plunger coupler 34 may be in the form of a shaft 36a that extends from a body of the syringe plunger 32, together with a head or button 36b. However, the syringe plunger coupler 34 may be of any appropriate size, shape, configuration, and/or type.

Generally, the syringe plunger drive assembly 14 of the power injector 10 may interact with the syringe plunger 32 of the syringe 28 in any appropriate manner (e.g., by mechanical contact; by an appropriate coupling (mechanical or otherwise)) so as to be able to move or advance the syringe plunger 32 (relative to the syringe barrel 30) in at least one direction (e.g., to discharge fluid from the corresponding syringe 28). That is, although the syringe plunger drive assembly 14 may be capable of bi-directional motion (e.g., via operation of the same drive source 16), the power injector 10 may be configured such that the operation of the syringe plunger drive assembly 14 actually only moves each syringe plunger 32 being used by the power injector 10 in only one direction. However, the syringe plunger drive assembly 14 may be configured to interact with each syringe plunger 32 being used by the power injector 10 so as to be able to move each such syringe plunger 32 in each of two different directions (e.g. in different directions along a common axial path).

Retraction of the syringe plunger 32 may be utilized to accommodate a loading of fluid into the syringe barrel 30 for a subsequent injection or discharge, may be utilized to actually draw fluid into the syringe barrel 30 for a subsequent injection or discharge, or for any other appropriate purpose. Certain configurations may not require that the syringe plunger drive assembly 14 be able to retract the syringe plunger 32, in which case the ram coupler 22 and syringe plunger coupler 34 may not be desired. In this case, the syringe plunger drive assembly 14 may be retracted for purposes of executing another fluid delivery operation (e.g., after another pre-filled syringe 28 has been installed). Even when a ram coupler 22 and syringe plunger coupler 34 are utilized, these components may or may not be coupled when the ram 20 advances the syringe plunger 32 to discharge fluid from the syringe 28 (e.g., the ram 20 may simply “push on” the syringe plunger coupler 34 or directly on a proximal end of the syringe plunger 32). Any single motion or combination of motions in any appropriate dimension or combination of dimensions may be utilized to dispose the ram coupler 22 and syringe plunger coupler 34 in a coupled state or condition, to dispose the ram coupler 22 and syringe plunger coupler 34 in an un-coupled state or condition, or both.

The syringe 28 may be installed on the powerhead 12 in any appropriate manner. For instance, the syringe 28 could be configured to be installed directly on the powerhead 12. In the illustrated embodiment, a housing 24 is appropriately mounted on the powerhead 12 to provide an interface between the syringe 28 and the powerhead 12. This housing 24 may be in the form of an adapter to which one or more configurations of syringes 28 may be installed, and where at least one configuration for a syringe 28 could be installed directly on the powerhead 12 without using any such adapter. The housing 24 may also be in the form of a faceplate to which one or more configurations of syringes 28 may be installed. In this case, it may be such that a faceplate is required to install a syringe 28 on the powerhead 12—the syringe 28 could not be installed on the powerhead 12 without the faceplate. When a pressure jacket 26 is being used, it may be installed on the powerhead 12 in the various manners discussed herein in relation to the syringe 28, and the syringe 28 will then thereafter be installed in the pressure jacket 26.

The housing 24 may be mounted on and remain in a fixed position relative to the powerhead 12 when installing a syringe 28. Another option is to movably interconnect the housing 24 and the powerhead 12 to accommodate installing a syringe 28. For instance, the housing 24 may move within a plane that contains the double-headed arrow A to provide one or more of coupled state or condition and an un-coupled state or condition between the ram coupler 22 and the syringe plunger coupler 34.

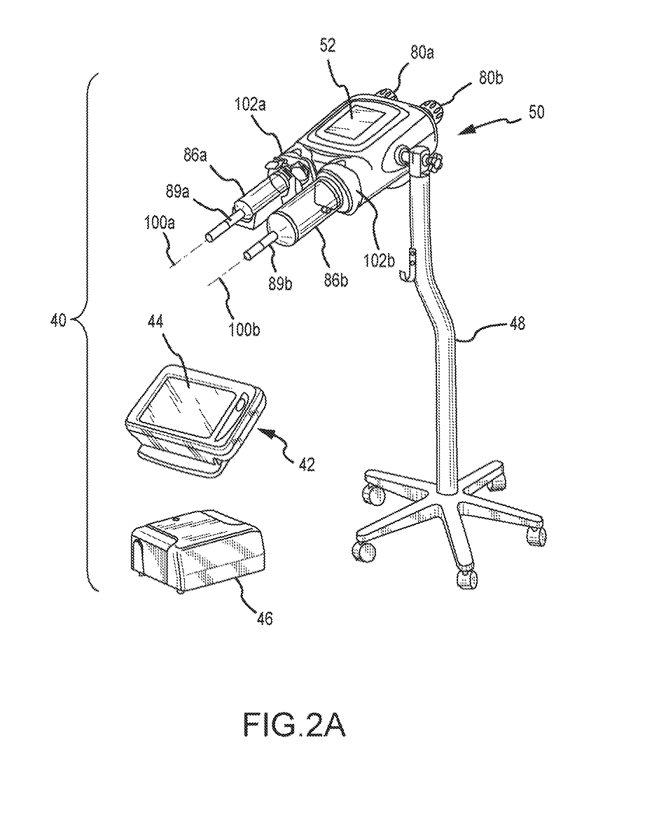

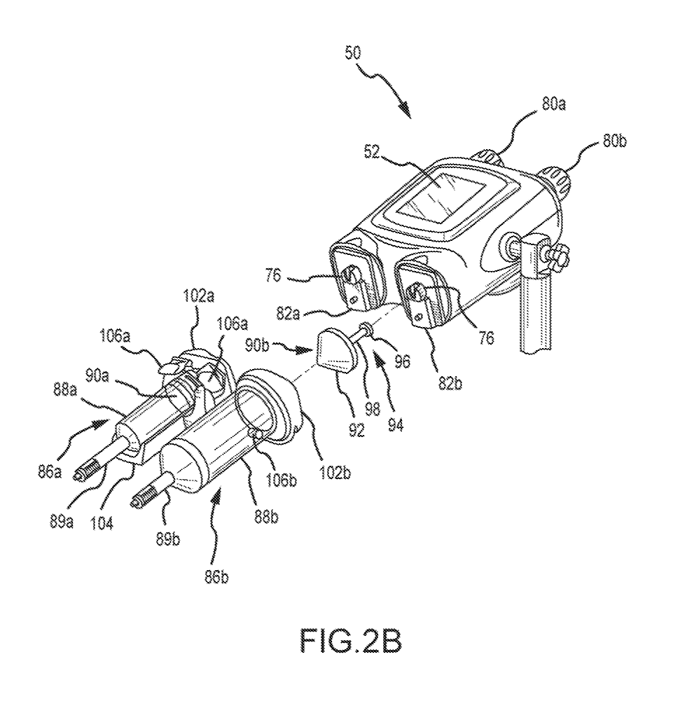

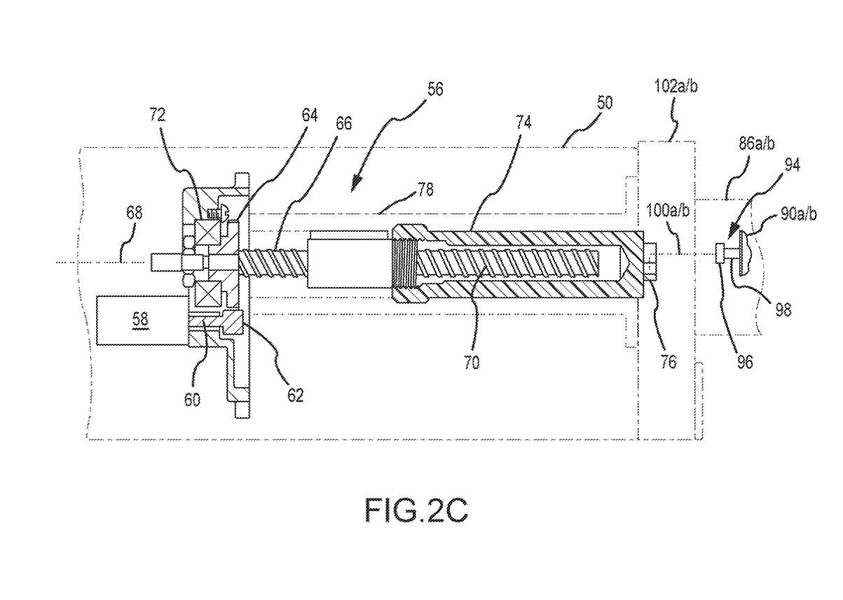

One particular power injector configuration is illustrated in FIG. 2A, is identified by a reference numeral 40, and is at least generally in accordance with the power injector 10 of FIG. 1. The power injector 40 includes a powerhead 50 that is mounted on a portable stand 48. A pair of syringes 86a, 86b for the power injector 40 are mounted on the powerhead 50. Fluid may be discharged from the syringes 86a, 86b during operation of the power injector 40.

The portable stand 48 may be of any appropriate size, shape, configuration, and/or type. Wheels, rollers, casters, or the like may be utilized to make the stand 48 portable. The powerhead 50 could be maintained in a fixed position relative to the portable stand 48. However, it may be desirable to allow the position of the powerhead 50 to be adjustable relative to the portable stand 48 in at least some manner. For instance, it may be desirable to have the powerhead 50 in one position relative to the portable stand 48 when loading fluid into one or more of the syringes 86a, 86b, and to have the powerhead 50 in a different position relative to the portable stand 48 for performance of an injection procedure. In this regard, the powerhead 50 may be movably interconnected with the portable stand 48 in any appropriate manner (e.g., such that the powerhead 50 may be pivoted through at least a certain range of motion, and thereafter maintained in the desired position).

It should be appreciated that the powerhead 50 could be supported in any appropriate manner for providing fluid. For instance, instead of being mounted on a portable structure, the powerhead 50 could be interconnected with a support assembly, that in turn is mounted to an appropriate structure (e.g., ceiling, wall, floor). Any support assembly for the powerhead 50 may be positionally adjustable in at least some respect (e.g., by having one or more support sections that may be repositioned relative to one or more other support sections), or may be maintained in a fixed position. Moreover, the powerhead 50 may be integrated with any such support assembly so as to either be maintained in a fixed position or so as to be adjustable relative the support assembly.

The powerhead 50 includes a graphical user interface or GUI 52. This GUI 52 may be configured to provide one or any combination of the following functions: controlling one or more aspects of the operation of the power injector 40; inputting/editing one or more parameters associated with the operation of the power injector 40; and displaying appropriate information (e.g., associated with the operation of the power injector 40). The power injector 40 may also include a console 42 and powerpack 46 that each may be in communication with the powerhead 50 in any appropriate manner (e.g., via one or more cables), that may be placed on a table or mounted on an electronics rack in an examination room or at any other appropriate location, or both. The powerpack 46 may include one or more of the following and in any appropriate combination: a power supply for the injector 40; interface circuitry for providing communication between the console 42 and powerhead 50; circuitry for permitting connection of the power injector 40 to remote units such as remote consoles, remote hand or foot control switches, or other original equipment manufacturer (OEM) remote control connections (e.g., to allow for the operation of power injector 40 to be synchronized with the x-ray exposure of an imaging system); and any other appropriate componentry. The console 42 may include a touch screen display 44, which in turn may provide one or more of the following functions and in any appropriate combination: allowing an operator to remotely control one or more aspects of the operation of the power injector 40; allowing an operator to enter/edit one or more parameters associated with the operation of the power injector 40; allowing an operator to specify and store programs for automated operation of the power injector 40 (which can later be automatically executed by the power injector 40 upon initiation by the operator); and displaying any appropriate information relation to the power injector 40 and including any aspect of its operation.

Various details regarding the integration of the syringes 86a, 86b with the powerhead 50 are presented in FIG. 2B. Each of the syringes 86a, 86b includes the same general components. The syringe 86a includes plunger or piston 90a that is movably disposed within a syringe barrel 88a. Movement of the plunger 90a along an axis 100a (FIG. 2A) via operation of the powerhead 50 will discharge fluid from within a syringe barrel 88a through a nozzle 89a of the syringe 86a. An appropriate conduit (not shown) will typically be fluidly interconnected with the nozzle 89a in any appropriate manner to direct fluid to a desired location (e.g., a patient). Similarly, the syringe 86b includes plunger or piston 90b that is movably disposed within a syringe barrel 88b. Movement of the plunger 90b along an axis 100b (FIG. 2A) via operation of the powerhead 50 will discharge fluid from within the syringe barrel 88b through a nozzle 89b of the syringe 86b. An appropriate conduit (not shown) will typically be fluidly interconnected with the nozzle 89b in any appropriate manner to direct fluid to a desired location (e.g., a patient).

The syringe 86a is interconnected with the powerhead 50 via an intermediate faceplate 102a. This faceplate 102a includes a cradle 104 that supports at least part of the syringe barrel 88a, and which may provide/accommodate any additional functionality or combination of functionalities. A mounting 82a is disposed on and is fixed relative to the powerhead 50 for interfacing with the faceplate 102a. A ram coupler 76 of a ram 74 (FIG. 2C), which are each part of a syringe plunger drive assembly or syringe plunger driver 56 (FIG. 2C) for the syringe 86a, is positioned in proximity to the faceplate 102a when mounted on the powerhead 50. Details regarding the syringe plunger drive assembly 56 will be discussed in more detail below in relation to FIG. 2C. Generally, the ram coupler 76 may be coupled with the syringe plunger 90a of the syringe 86a, and the ram coupler 76 and ram 74 (FIG. 2C) may then be moved relative to the powerhead 50 to move the syringe plunger 90a along the axis 100a (FIG. 2A). It may be such that the ram coupler 76 is engaged with, but not actually coupled to, the syringe plunger 90a when moving the syringe plunger 90a to discharge fluid through the nozzle 89a of the syringe 86a.

The faceplate 102a may be moved at least generally within a plane that is orthogonal to the axes 100a, 100b (associated with movement of the syringe plungers 90a, 90b, respectively, and illustrated in FIG. 2A), both to mount the faceplate 102a on and remove the faceplate 102a from its mounting 82a on the powerhead 50. The faceplate 102a may be used to couple the syringe plunger 90a with its corresponding ram coupler 76 on the powerhead 50. In this regard, the faceplate 102a includes a pair of handles 106a. Generally and with the syringe 86a being initially positioned within the faceplate 102a, the handles 106a may be moved to in turn move/translate the syringe 86a at least generally within a plane that is orthogonal to the axes 100a, 100b (associated with movement of the syringe plungers 90a, 90b, respectively, and illustrated in FIG. 2A). Moving the handles 106a to one position moves/translates the syringe 86a (relative to the faceplate 102a) in an at least generally downward direction to couple its syringe plunger 90a with its corresponding ram coupler 76. Moving the handles 106a to another position moves/translates the syringe 86a (relative to the faceplate 102a) in an at least generally upward direction to uncouple its syringe plunger 90a from its corresponding ram coupler 76.

The syringe 86b is interconnected with the powerhead 50 via an intermediate faceplate 102b. A mounting 82b is disposed on and is fixed relative to the powerhead 50 for interlacing with the faceplate 102b. A ram coupler 76 of a ram 74 (FIG. 2C), which are each part of a syringe plunger drive assembly 56 for the syringe 86b, is positioned in proximity to the faceplate 102b when mounted to the powerhead 50. Details regarding the syringe plunger drive assembly 56 again will be discussed in more detail below in relation to FIG. 2C. Generally, the ram coupler 76 may be coupled with the syringe plunger 90b of the syringe 86b, and the ram coupler 76 and ram 74 (FIG. 2C) may be moved relative to the powerhead 50 to move the syringe plunger 90b along the axis 100b (FIG. 2A). It may be such that the ram coupler 76 is engaged with, but not actually coupled to, the syringe plunger 90b when moving the syringe plunger 90b to discharge fluid through the nozzle 89b of the syringe 86b.

The faceplate 102b may be moved at least generally within a plane that is orthogonal to the axes 100a, 100b (associated with movement of the syringe plungers 90a, 90b, respectively, and illustrated in FIG. 2A), both to mount the faceplate 102b on and remove the faceplate 102b from its mounting 82b on the powerhead 50. The faceplate 102b also may be used to couple the syringe plunger 90b with its corresponding ram coupler 76 on the powerhead 50. In this regard, the faceplate 102b may include a handle 106b. Generally and with the syringe 86b being initially positioned within the faceplate 102b, the syringe 86b may be rotated along its long axis 100b (FIG. 2A) and relative to the faceplate 102b. This rotation may be realized by moving the handle 106b, by grasping and turning the syringe 86b, or both. In any case, this rotation moves/translates both the syringe 86b and the faceplate 102b at least generally within a plane that is orthogonal to the axes 100a, 100b (associated with movement of the syringe plungers 90a, 90b, respectively, and illustrated in FIG. 2A). Rotating the syringe 86b in one direction moves/translates the syringe 86b and faceplate 102b in an at least generally downward direction to couple the syringe plunger 90b with its corresponding ram coupler 76. Rotating the syringe 86b in the opposite direction moves/translates the syringe 86b and faceplate 102b in an at least generally upward direction to uncouple its syringe plunger 90b from its corresponding ram coupler 76.

As illustrated in FIG. 2B, the syringe plunger 90b includes a plunger body 92 and a syringe plunger coupler 94. This syringe plunger coupler 94 includes a shaft 98 that extends from the plunger body 92, along with a head 96 that is spaced from the plunger body 92. Each of the ram couplers 76 includes a larger slot that is positioned behind a smaller slot on the face of the ram coupler 76. The head 96 of the syringe plunger coupler 94 may be positioned within the larger slot of the ram coupler 76, and the shaft 98 of the syringe plunger coupler 94 may extend through the smaller slot on the face of the ram coupler 76 when the syringe plunger 90b and its corresponding ram coupler 76 are in a coupled state or condition. The syringe plunger 90a may include a similar syringe plunger coupler 94 for interfacing with its corresponding ram coupler 76.

The powerhead 50 is utilized to discharge fluid from the syringes 86a, 86b in the case of the power injector 40. That is, the powerhead 50 provides the motive force to discharge fluid from each of the syringes 86a, 86b. One embodiment of what may be characterized as a syringe plunger drive assembly or syringe plunger driver is illustrated in FIG. 2C, is identified by reference numeral 56, and may be utilized by the powerhead 50 to discharge fluid from each of the syringes 86a, 86b. A separate syringe plunger drive assembly 56 may be incorporated into the powerhead 50 for each of the syringes 86a, 86b. In this regard and referring back to FIGS. 2A-B, the powerhead 50 may include hand-operated knobs 80a and 80b for use in separately controlling each of the syringe plunger drive assemblies 56.

Initially and in relation to the syringe plunger drive assembly 56 of FIG. 2C, each of its individual components may be of any appropriate size, shape, configuration and/or type. The syringe plunger drive assembly 56 includes a motor 58, which has an output shaft 60. A drive gear 62 is mounted on and rotates with the output shaft 60 of the motor 58. The drive gear 62 is engaged or is at least engageable with a driven gear 64. This driven gear 64 is mounted on and rotates with a drive screw or shaft 66. The axis about which the drive screw 66 rotates is identified by reference numeral 68. One or more bearings 72 appropriately support the drive screw 66.

A carriage or ram 74 is movably mounted on the drive screw 66. Generally, rotation of the drive screw 66 in one direction axially advances the ram 74 along the drive screw 66 (and thereby along axis 68) in the direction of the corresponding syringe 86a/b, while rotation of the drive screw 66 in the opposite direction axially advances the ram 74 along the drive screw 66 (and thereby along axis 68) away from the corresponding syringe 86a/b. In this regard, the perimeter of at least part of the drive screw 66 includes helical threads 70 that interface with at least part of the ram 74. The ram 74 is also movably mounted within an appropriate bushing 78 that does not allow the ram 74 to rotate during a rotation of the drive screw 66. Therefore, the rotation of the drive screw 66 provides for an axial movement of the ram 74 in a direction determined by the rotational direction of the drive screw 66.

The ram 74 includes a coupler 76 that that may be detachably coupled with a syringe plunger coupler 94 of the syringe plunger 90a/b of the corresponding syringe 86a/b. When the ram coupler 76 and syringe plunger coupler 94 are appropriately coupled, the syringe plunger 90a/b moves along with ram 74. FIG. 2C illustrates a configuration where the syringe 86a/b may be moved along its corresponding axis 100a/b without being coupled to the ram 74. When the syringe 86a/b is moved along its corresponding axis 100a/b such that the head 96 of its syringe plunger 90a/b is aligned with the ram coupler 76, but with the axes 68 still in the offset configuration of FIG. 2C, the syringe 86a/b may be translated within a plane that is orthogonal to the axis 68 along which the ram 74 moves. This establishes a coupled engagement between the ram coupler 76 and the syringe plunger coupler 96 in the above-noted manner.

The power injectors 10, 40 of FIGS. 1 and 2A-C each may be used for any appropriate application, including without limitation for medical imaging applications where fluid is injected into a subject (e.g., a patient). Representative medical imaging applications for the power injectors 10, 40 include without limitation computed tomography or CT imaging, magnetic resonance imaging or MRI, single photon emission computed tomography or SPECT imaging, positron emission tomography or PET imaging, X-ray imaging, angiographic imaging, optical imaging, and ultrasound imaging. The power injectors 10, 40 each could be used alone or in combination with one or more other components. The power injectors 10, 40 each may be operatively interconnected with one or more components, for instance so that information may be conveyed between the power injector 10, 40 and one or more other components (e.g., scan delay information, injection start signal, injection rate).

Any number of syringes may be utilized by each of the power injectors 10, 40, including without limitation single-head configurations (for a single syringe) and dual-head configurations (for two syringes). In the case of a multiple syringe configuration, each power injector 10, 40 may discharge fluid from the various syringes in any appropriate manner and according to any timing sequence (e.g., sequential discharges from two or more syringes, simultaneous discharges from two or more syringes, or any combination thereof). Multiple syringes may discharge into a common conduit (e.g., for provision to a single injection site), or one syringe may discharge into one conduit (e.g., for provision to one injection site), while another syringe may discharge into a different conduit (e.g., for provision to a different injection site). Each such syringe utilized by each of the power injectors 10, 40 may include any appropriate fluid (e.g., a medical fluid), for instance contrast media, a radiopharmaceutical, saline, and any combination thereof. Each such syringe utilized by each of the power injectors 10, 40 may be installed in any appropriate manner (e.g., rear-loading configurations may be utilized; front-loading configurations may be utilized; side-loading configurations may be utilized).

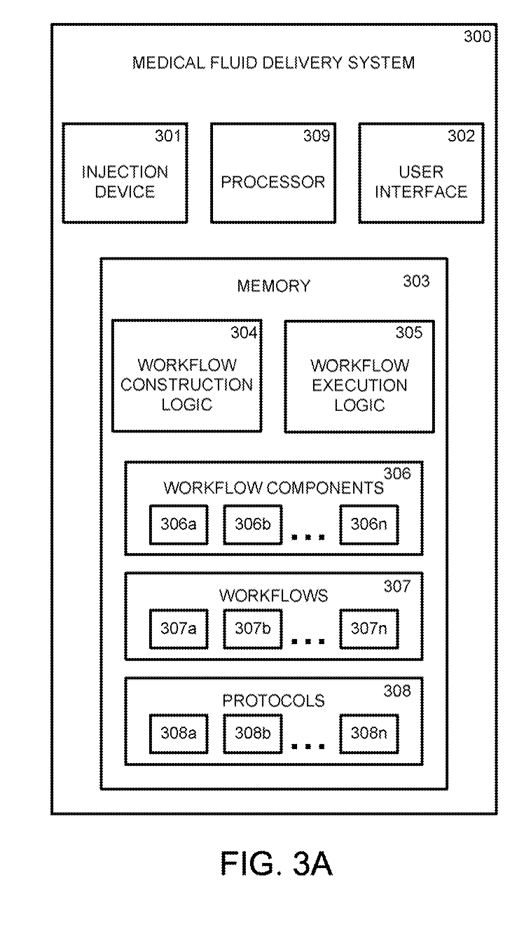

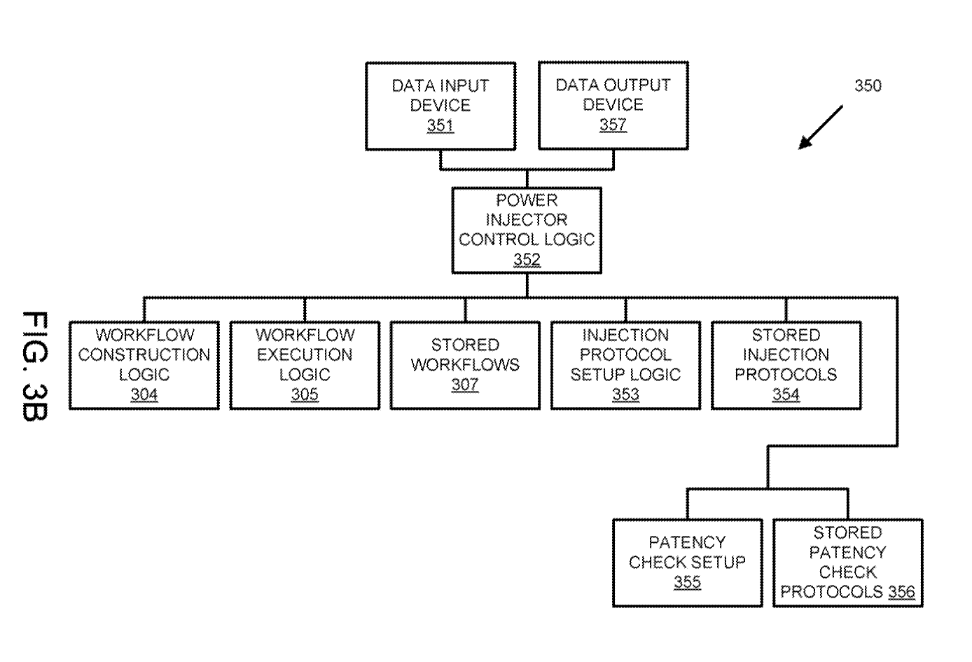

FIG. 3A is a block diagram of a medical fluid delivery system 300 that includes workflow construction logic 304, workflow execution logic 305, and a processor 309. The processor 309 may be operable to, inter alia, execute the workflow construction logic 304 and workflow execution logic 305. Generally, FIG. 3A illustrates components related to workflow usage and management. Accordingly, the medical fluid delivery system 300 may include various other components that are not illustrated in FIG. 3A. The processor 309 may be located in any appropriate location or distributed between any appropriate locations within the medical fluid delivery system 300. The medical fluid delivery system 300 may include a single workflow (such as workflow 307a) or a plurality of workflows 307 (e.g., workflow 307a through 307n, where n is any appropriate number). As used herein, a “workflow” is a set of workflow components arranged in a predetermined order that when executed are able to be used to complete a procedure using the medical fluid delivery system 300. Such a procedure may, for example, include setting up the medical fluid delivery system 300 and then injecting a patient with medical fluid (e.g., contrast media and/or saline) with the medical fluid delivery system 300 in conjunction with an imaging procedure.

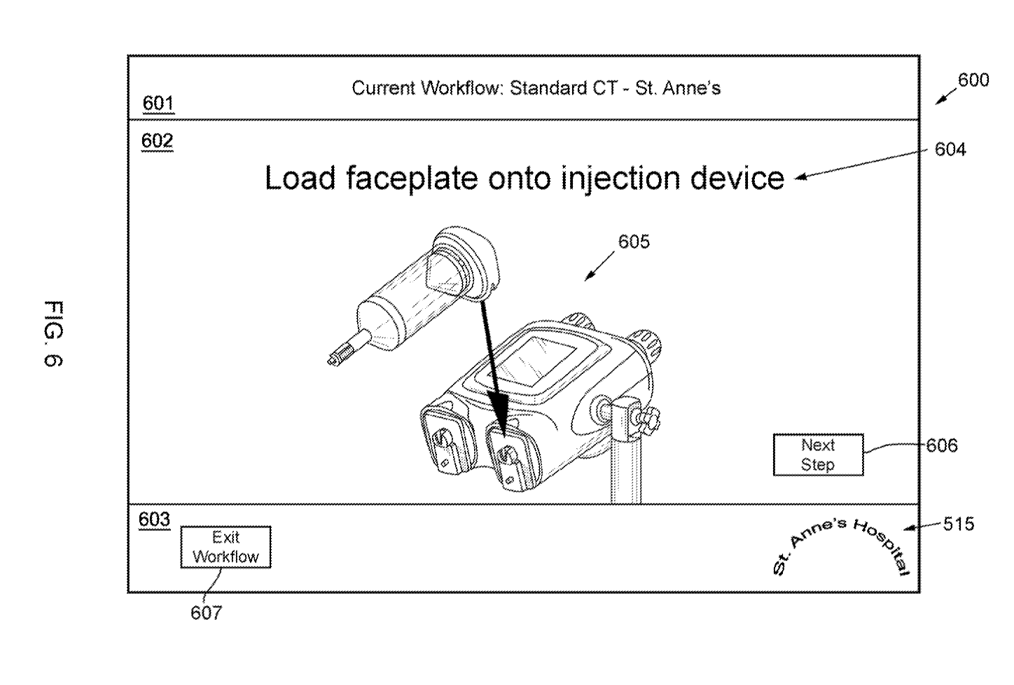

A “workflow component” is a discrete task or set of tasks that may be performed in connection with the use of the medical fluid delivery system 300. The medical fluid delivery system 300 may include a plurality of workflow components 306 (e.g., workflow component 306a through 306n, where n is any appropriate number). In this regard, the plurality of workflow components 306 may be considered to be a set of available steps or procedural building blocks for use in constructing a workflow. For example, workflow component 306a may include instructions to load or mount a prefilled syringe onto an injection device 301 (see also FIG. 6 discussed below). In another example, workflow component 306a may include a set of instructions related to performance of a patency check. A workflow component may be a task or set of tasks (e.g., load a syringe, connect tubing, verify patient information) that are to be performed by a user during the performance of a workflow. The term “user,” as used herein, may be an administrator, clinician, doctor or any other appropriate person associated with configuring the medical fluid delivery system 300 and/or administering a medical fluid using the medical fluid delivery system 300. A workflow component may be a task or set of tasks that are to be performed by the medical fluid delivery system 300 (e.g., purge air, inject medical fluid into a patient) during the performance of a workflow. A workflow component may also be a task or set of tasks that are to be performed by a combination of the user and the medical fluid delivery system 300. Examples of tasks that may be associated with workflow components include syringe type selection, syringe quantity selection, injection protocol selection, syringe loading, syringe re-loading, syringe filling, tubing set connecting and purging, patency check, test injection, main injection, results display, results printing, and tubing and syringe removal. Examples of items that may vary from workflow to workflow include syringe type (prefill or empty disposable), saline use (if saline is used, both heads of a dual head injection device may be utilized), whether or not a patency check is to be performed, whether or not a test injection is to be performed, whether or not to display results, how and where to display results, whether or not to print results, and the order of tubing and syringe removal.

Generally, the workflow construction logic 304 may be used to construct a workflow, such as workflow 307a. Once the workflow 307a for a particular procedure has been constructed (e.g., created, designed), it may be saved and retrieved. The workflow execution logic 305 may then be used to execute the workflow 307a. In this regard, the workflow execution logic 305 may initiate functionality of the medical fluid delivery system 300. Once the workflow execution logic 305 executes the workflow 307a, the medical fluid delivery system 300 may present to the user (e.g., through the user interface 302) each workflow component sequentially according to the workflow 307a. Each workflow component may include a simplified output (e.g., a display or output on the user interface 302) to assist the user in completing a particular task or set of tasks related to the workflow component. After completion of each workflow component, the workflow execution logic 305 may automatically advance to the next workflow component and guide the user through performing that workflow component. In this regard, the user interface 302 may present to the user reduced complexity relative to the complexity typically associated with prior art power injectors. By presenting the individual workflow components of the workflow 307a in a predetermined order, the potential for deviation from the workflow 307a is reduced. Furthermore, training requirements for users may be reduced due to the simplified user interface 302 and predetermined workflow component sequence.

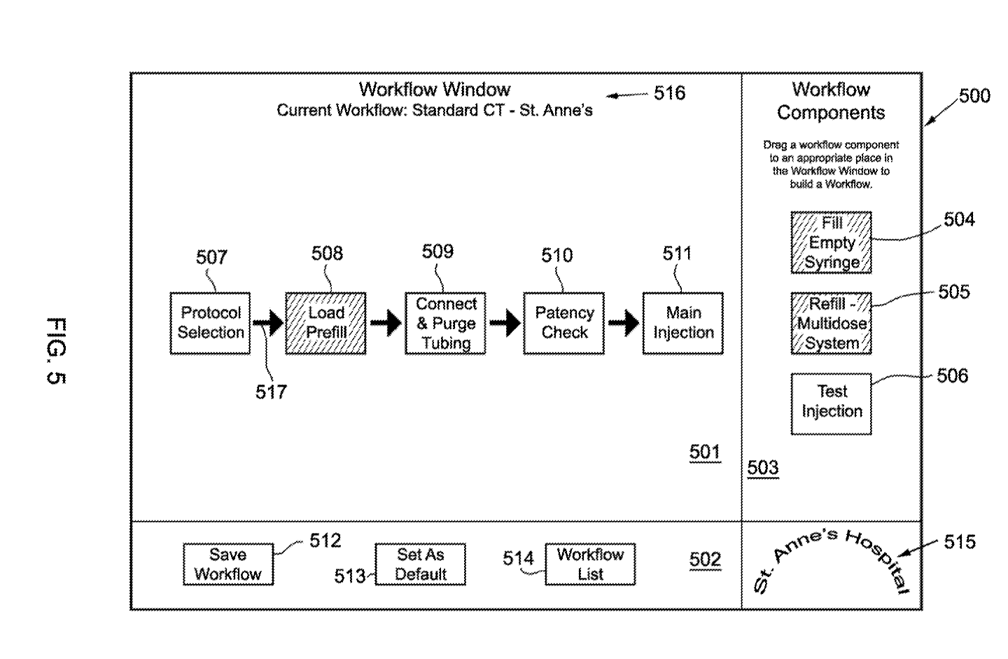

The medical fluid delivery system 300 may include a single protocol (such as protocol 308a) or a plurality of protocols 308 (e.g., protocol 308a through protocol 308n, where n is any appropriate number). A workflow of the plurality of workflows 307 may include and/or be associated with a single protocol (such as protocol 308a) or two or more protocols of the plurality of protocols 308. For example, workflow 307a may include injection protocol 308a that is comprised of a set of instructions to be followed by the injection device 301 when injecting medical fluid into a patient. Injection protocol 308a may include, for example, target fluid pressure and/or target fluid flow rates to be achieved at various points while injecting medical fluid into a patient. In another arrangement, workflow 307a may include a protocol selection workflow component 507 (see FIG. 5) where a protocol is selected from the plurality of protocols 308.

The workflow construction logic 304, workflow execution logic 305, plurality of workflow components 306, plurality of workflows 307, and plurality of protocols 308 may be stored within a memory 303 of the medical fluid delivery system 300. The memory unit 303 may be single device (e.g., a hard drive located within the medical fluid delivery system 300) or it may be comprised of several independent devices located at a plurality of locations. For example, the plurality of protocols 308 may be stored in a RAM (random access memory) module disposed within the injection device 301, while the plurality of workflow components 306 may be stored remotely and accessible to the injection device 301 via a network connection.

The injection device 301 may be in the form of the powerhead 50 discussed above with reference to FIGS. 2A and 2B. The medical fluid delivery system 300 may further include a user interface 302. The user interface 302 may be in the form of one or more components operable to receive input from a user and to display an output for a user. For example, the user interface 302 may be in the form of the GUI 52 on the powerhead 50. The user interface 302 may be in the form of any appropriate device or devices for accepting inputs and producing outputs. The user interface 302 may, for example, be in the form of a touch screen operable to receive tactile input from a user and display an output. In another example, the user interface 302 may be in the form of a keyboard and monitor.

Generally, the workflow construction logic 304 may be used by an administrator to construct a workflow, such as workflow 307a, from the plurality of workflow components 306. The “administrator” may be any appropriate person or group responsible for specifying the procedures and steps to be followed while administering medical fluid with the medical fluid delivery system 300 by a particular entity. The assembled workflow 307a may be stored in the memory 303. Other workflows of the plurality of workflows 307 may also be constructed from the plurality of workflow components 306. Each workflow of the plurality of workflows 307 may include a proper subset of workflow components from the plurality of workflow components 306. As used herein, a second set is a “proper subset” of a first set if every element in the second set is in the first set and the first set has some elements which are not in the second set. Accordingly, a proper subset of workflow components, as used herein, is a subset of the plurality of workflow components 306 where not every workflow component of the plurality of workflow components 306 is included in the proper subset. The workflow 307a may be constructed in accordance with the practices of the institution or entity responsible for administration of the medical fluids. For example, a particular hospital may construct the workflow 307a to comply with their practice.

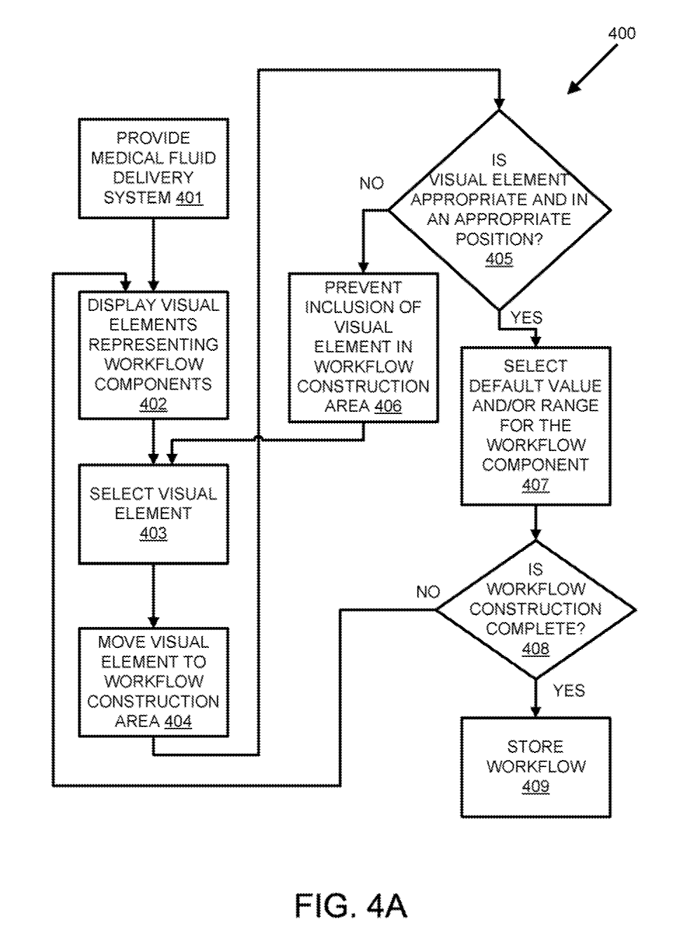

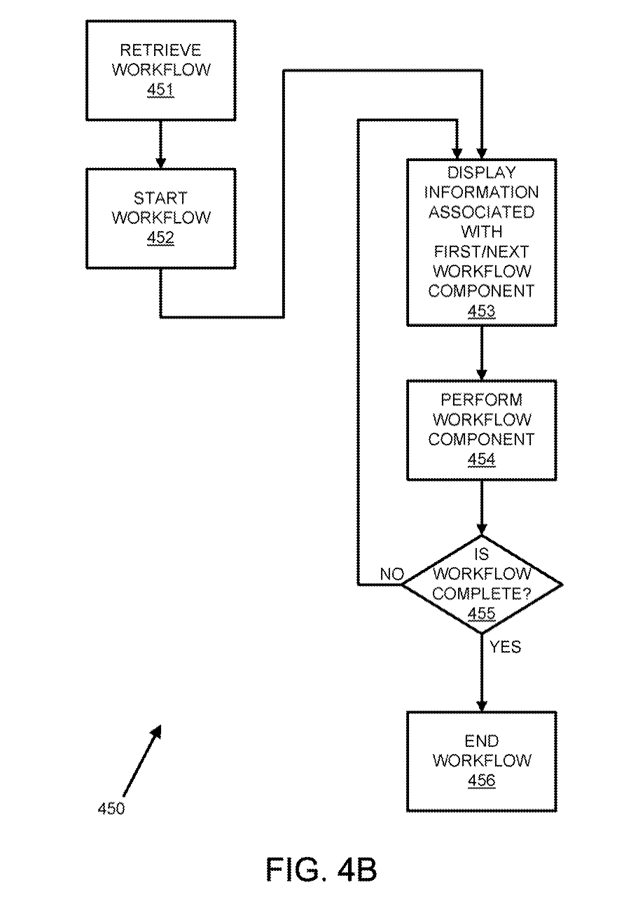

After a workflow, such as workflow 307a, is constructed and stored in the memory 303, it may be accessed by a user by selecting workflow 307a and running the workflow execution logic 305 to execute workflow 307a. The workflow execution logic 305 may step through the workflow components included within workflow 307a to guide the user through the process of setting up the medical fluid delivery system 300 and/or injecting a medical fluid into a patient (e.g., as described below with reference to FIGS. 4A through 6).

FIG. 3B is an alternate way of characterizing the medical fluid delivery system 300 of FIG. 3A. In the characterization of FIG. 3B, a medical fluid delivery system 350 includes a data input device 351 that may provide inputs to power injector control logic 352. The data input device 351 may be a touch screen, keypad, or any other appropriate device for inputting data. The power injector control logic 352 may provide high level functionality for the medical fluid delivery system 350, including receiving data input from the data input device 351 and providing visual output to a user (e.g., though the data input device 351 where the data input device 351 is a touch screen or through a data output device 357). An administrator may use the power injector control logic 352 to initiate workflow construction logic 304 which may in turn be used to generate a workflow such as workflow 307a. Workflow 307a may then be stored along with any other stored workflows 307. When the medical fluid delivery system 350 is to be used to inject medical fluid into a patient, a user may access one of the stored workflows 307 and execute the workflow using the workflow execution logic 305.

A workflow may include executing injection protocol setup logic 353 that may be used to select and execute a stored injection protocol from a plurality of stored injection protocols 354. The medical fluid delivery system 350 may also include patency check setup logic 355 that may be used to select and execute a stored patency check protocol from a plurality of stored patency check protocols 356.

FIG. 4A is a flow diagram 400 of one embodiment of a method of constructing a workflow. The first step 401 may be to provide a medical fluid delivery system such as the medical fluid delivery system 300 described with reference to FIG. 3A. The next step 402 may be to display visual elements representing workflow components. FIG. 5 is an illustration of an exemplary workflow construction display 500 that may be displayed on the user interface 302 of the medical fluid delivery system 300 of FIG. 3A during construction of a workflow. The visual elements may be displayed as workflow components in a workflow component area 503. In FIG. 5, three visual elements representing workflow components are illustrated: a fill empty syringe workflow component 504, a refill multidose workflow component 505, and a test injection workflow component 506. Such visual elements may be referred to herein as workflow components in place of reciting that they are visual elements that represent corresponding workflow components. In other implementations, more or fewer workflow components may be displayed in the workflow component area 503. Moreover, the workflow components that are displayed in the workflow component area 503 may be context sensitive in that selection of a particular workflow component may cause other workflow components not to be displayed in the workflow component area 503. For example, in FIG. 5 a “Load PrefiII” 508 workflow component has been included as a selected workflow in a workflow construction area 501 (described below). Accordingly, workflow components associated with filling empty syringes and exclusive to injection processes that use bulk fluid containers as the medical fluid source may not be displayed while the “Load Prefill” 508 workflow component is in the workflow construction area 501.

Continuing to collectively refer to FIGS. 4 A and 5, following the display of visual elements representing workflow components, a next step 403 may be to select one or more of the displayed visual elements for inclusion in the workflow under construction. Selecting a displayed visual element may take any appropriate form including, for example, touching a region of a touch screen corresponding to the visual element to be selected, pressing a button along the periphery of a display that corresponds to a visual element on the display, and/or using a mouse to click on the visual element. Once a visual element is selected, the next step 404 may be to move the visual element to the workflow construction area 501. Moving the visual element may take the form of dragging and dropping the visual element using known icon manipulation techniques such as, for example, sliding a mouse pointer or finger across the workflow construction display 500. A user may use such movements to place the visual element in a desired location relative to other workflow components located within the workflow construction area 501. Alternatively, by selecting the visual element in step 403, the workflow construction logic 304 may automatically move the selected visual element into the workflow construction area 501 at a location determined appropriate by the workflow construction logic 304. A user may then reposition the visual element.

When a user positions the workflow component in the workflow construction area 501, the workflow construction logic 304 may make a determination as to whether or not the workflow component is appropriate for the workflow being constructed and whether or not the workflow component is in an appropriate position, such that the resultant workflow or portion thereof is safe and clinically appropriate. If the workflow construction logic 304 makes the determination that the workflow component would result in a workflow that is not safe and clinically appropriate or that the workflow component is an inappropriate position, the workflow construction logic 304 may take the step 406 of preventing the inclusion of the visual element in the workflow construction area 501. Such prevention may take the form of moving the visual element back to the workflow component area 503. Alternatively, the workflow construction logic 304 may produce alerts letting the administrator know of the potentially unsafe and/or clinically inappropriate workflow. The workflow construction logic 304 may allow the administrator to override such alerts in at least some alert generating situations.

If the workflow construction logic 304 makes the determination that the workflow component has been appropriately placed, the next step 407 may be to prompt the user to select a default value and/or range of allowable/suggested values for the workflow component. In situations where no value and/or range is needed for the workflow component, step 407 may of course be skipped.

For explanatory purposes, a scenario where a default value is entered will now be described. In constructing a workflow, an administrator may move the “Load Prefill” 508 workflow component into the workflow construction area 501. After such an action, the workflow construction logic 304 may query the administrator as to whether or not the administrator would like to enter a default value for the “Load Prefill” 508 workflow component. In response, the administrator may enter a default value (e.g., 125 milliliters) if the administrator has made the decision to typically use a 125 milliliter prefilled syringe when making medical fluid injections using the medical fluid delivery system 300. In such a scenario, during workflow execution, the workflow execution logic 305 may prompt a user to install a 125 milliliter syringe. Alternatively, the administrator may decline to enter a default value, in which case when the “Load Prefill” 508 workflow component is enacted during workflow execution, the workflow execution logic 305 may prompt the user to enter in the volume of the prefilled syringe that has been loaded or is to be loaded. Similarly, in a step where a user may have the ability to enter any value, a range may be specified in step 407 such that if the user enters a value outside of the range during workflow execution, the workflow execution logic 305 may prevent entering such a value, sound and/or display an alarm to the user indicating that they have entered a value outside of an expected and/or allowed range, and/or perform some other appropriate action.

The next step 408 may be to determine if the workflow construction process is complete. Such a determination may be made by the user and/or the workflow construction logic 304. If the workflow construction process is not complete, the process may return to step 402 where the visual elements are displayed, and another visual element may be selected and incorporated into the workflow. If the workflow construction is complete, the next step 409 may be to store the workflow. This may include giving the workflow a name or entering other identification parameters into the medical fluid delivery system 300.

Turning to FIG. 5, the workflow construction display 500 may also include a workflow management area 502. The workflow management area 502 may include a “Save Workflow” button 512, a “Set As Default” button 513, and a “Workflow List” button 514. These buttons 512-514 may be used to manage the workflows stored in the memory 303 of the medical fluid delivery system 300 (FIG. 3A). For example, the administrator may press the “Save Workflow” button 512 to save the current workflow into the memory 303. As used herein, “pressing” a button on the workflow construction display 500 or on a workflow execution display 600 (FIG. 6, discussed below) may include: pressing on the display 500, 600 where the display is on a touch screen; moving a mouse pointer to the button and clicking on the item with the mouse; pressing a button adjacent to the display 500, 600 in the region of the button being pressed; or any other appropriate method for a user to indicate selection of an item on a display. Various workflows may be accessed for editing and/or review by pressing the “Workflow List” button 514. Such an action may bring up another window that may show a list of workflows currently stored in the memory 303. The “Set As Default” button 513 may be used to set the current workflow as the default workflow for the medical fluid delivery system 300.

The workflow construction logic 304 may be configured such that a password is required. The password may be required to access all or part of the functionality of the workflow construction logic 304. For example, in one arrangement, a password may be needed to access any portion of the workflow construction logic 304, In another example, a password may be required to alter an existing saved workflow and/or change the default workflow for the medical fluid delivery system 300. Similarly, the workflow execution logic 305 may be configured such that a password is required. The password may be required to access all or part of the functionality of the workflow execution logic 305.

The workflow construction area 501 may display a graphical representation of a particular workflow being constructed and/or edited. As illustrated within the workflow construction area 501, a current workflow is made up of workflow components: “Protocol Selection” 507, “Load Prefill” 508, “Connect & Purge Tubing” 509, “Patency Check” 510, and “Main Injection” 511. The workflow construction area 501 also includes a header 516 identifying the workflow in this example as “Standard CT—St. Anne's.” The workflow construction area 501 may also include arrows 517 or other appropriate indicators to communicate the relationship between the workflow components within the workflow construction area 501.

Subsets of workflow components may be color-coded and/or employ other types of visual indicators to communicate relationships between elements. For example, as illustrated in FIG. 5, the “Load Prefill” 508, “Fill Empty Syringe” 504 and “Refill—Multidose System” 505 workflow components all share a common background pattern indicating that they are related. They may be related in that they may be mutually exclusive components where only one of the three may be present in any particular workflow. This would be the case, for example, where a particular workflow is being performed with a single syringe. The workflow construction logic 304 may provide indications of the relationship in response to an administrator's actions. For example, if an administrator were to attempt to add the “Fill Empty Syringe” 504 workflow component to the workflow construction area 501 illustrated in FIG. 5, the workflow construction logic 304 may generate an alert window, or other appropriate indicator, informing the administrator that the “Fill Empty Syringe” 504 workflow component is incompatible with the “Load Prefill” 508 workflow component and whether the administrator would like to cancel the action or replace the “Load Prefill” 508 workflow component with the “Fill Empty Syringe” 504 workflow component. In this regard, the workflow construction logic 304 may be operable to prevent the simultaneous inclusion within the workflow construction area 501 and/or the workflow being constructed of more than one of such mutually exclusive components.

The workflow construction logic 304 may also allow the administrator to add and/or edit graphical and/or textual elements to customize the workflow construction display 500 and/or the workflow execution display 600 (FIG. 6, discussed below). As illustrated in FIG. 5, a logo 515 may be added to the workflow construction display 500. The logo 515 may be positioned in any appropriate location within the workflow construction display 500. Furthermore, the workflow construction logic 304 may provide the ability to customize text displayed by the workflow construction logic 304 during workflow construction and by the workflow execution logic 305 during various steps of workflow execution associated with the workflow components. This allows the administrator to adjust the terminology used by the medical fluid delivery system 300 during workflow construction and/or execution to match the terminology used by the particular organization operating the medical fluid delivery system 300. For example, some organizations may refer to a “patency check” as an “extravasation check.” The workflow construction logic 304 may allow an administrator to substitute the term “extravasation check” for “patency check” during workflow execution, thus causing the medical fluid delivery system 300 to present the term “extravasation check” as used by the organization.

FIG. 4B is a flow diagram 450 of one embodiment of a method of executing a workflow using the workflow execution logic 305. The first step 451 may be to retrieve a workflow. This may be accomplished by a user through interaction with the user interface 302. For example, the user may select a workflow from the plurality of workflows 307 stored in the memory 303 of the medical fluid delivery system 300. Once the workflow is retrieved, the next step 452 may be to start the workflow. The selection and starting steps may occur simultaneously. For example, a user may touch or otherwise select an icon or other element representing a workflow and in response, the medical fluid delivery system 300 may retrieve and start the workflow.

Once the workflow is started, the next step 453 may be for the workflow execution logic 305 to display information associated with the first workflow component. This may then be followed by the step 454 of performing the workflow component. After completion of a workflow component in step 454, the next step 455 may be to determine whether the workflow has been completed. If the workflow has not been completed, the method may return to step 453 and display information associated with the next workflow component and repeat steps 453, 454 and 455. The method may continue in this fashion until the workflow is completed. In this regard, the workflow execution logic 305 may sequentially execute each included workflow component in the predetermined sequence. If it is determined that the workflow has been completed in step 455, the method moves to step 456 to end the workflow.

The performance of the workflow component of step 454 may be performed by a user, by the medical fluid delivery system 300 initiated by the workflow execution logic 305, or by a combination of the user and the medical fluid delivery system 300.

For example, the “Load Prefill” 508 workflow component may be a step performed primarily by the user. As illustrated in FIG. 6, where a task is to be completed by the user, the user interface 302 may present a workflow execution display 600 that includes a workflow instruction area 602. As illustrated, the workflow instruction area 602 may include a graphical instruction 605 depicting how to load the prefilled syringe onto the injection device 301. While the graphical instruction 605 FIG. 6 shows an illustration of a prefilled syringe already loaded onto a removable face place and the face plate being installed on the injector head, other graphical illustrations may show other manners of loading an empty or prefilled syringe onto a power head of an injector (e.g., mounting a syringe on a syringe mount that is already part of (e.g., removably attached to, integral with, etc.) the injector head). Furthermore, the graphical instruction 605 may be animated, include video instruction and/or include a series of still images to aid in demonstrating the task to be performed by the user. The workflow instruction area 602 may also include a textual instruction 604 describing the task to be performed. When the user has completed the step described in the workflow instruction area 602, the user may press the “Next Step” button 606 to indicate to the workflow execution logic 305 to move to the next step in the current workflow component or to move to the next workflow component.

The workflow execution display 600 may also include a workflow description area 601 and a workflow management area 603. As illustrated, the workflow description area 601 may include an indication of the name of the workflow currently active. As illustrated, the workflow management area 603 may include an “Exit Workflow” button 607 (described below) and/or a logo 515. Any other appropriate information may also be displayed in the workflow description area 601 and/or the workflow management area 603 including, for example, current time, patient name, user name, and medical fluid type being injected. The administrator, through the workflow construction logic 304, may be able to choose what information is to be displayed within the workflow description area 601 and/or the workflow management area 603. In this regard any information or button may be displayed in any appropriate area of the workflow execution display 600. For example, the administrator may position the “Exit Workflow” button 607 within the workflow description area 601. Furthermore, in a given configuration, the workflow description area 601 and the workflow management area 603 may be combined into a single area (e.g. along the top or bottom of the workflow execution display 600). In another configuration, the entirety of the workflow execution display 600 may be occupied by the workflow instruction area 602 and the various elements described in connection with the workflow description area 601 and the workflow management area 603 may be superimposed over the workflow instruction area 602 (e.g., similar to how the “Nest Step” button 606 is disposed within the workflow instruction area 602). Any other appropriate method of displaying the various elements discussed herein may be incorporated into the workflow execution display 600.

The “Patency Check” 510 workflow component shown in FIG. 5 may be a step performed primarily by the medical fluid delivery system 300. After the tubing has been connected and purged in accordance with the “Connect & Purge Tubing” 509 workflow component, the medical fluid delivery system 300 may automatically execute the “Patency Check” 510 workflow component to execute a patency check using one of the stored patency check protocols 356 (FIG. 3B). This may entail the medical fluid delivery system 300 dispensing a small amount of medical fluid and measuring the resistance (e.g., pressure) encountered to determine if a catheter is positioned inside a vein or adjacent to it in the subcutaneous tissue (a condition where medical fluid is injected into the subcutaneous tissue being commonly referred to as extravasation or infiltration). While the workflow execution logic 305 is performing the patency check, the user interface 302 may display an indication that the medical fluid delivery system 300 is currently performing a task. Such a display may include textual indications such as “Patency Check In Progress” and/or graphical indications such as a status bar reporting the percent completion of the patency check. When the workflow execution logic 305 completes the “Patency Check” 510 workflow component, the workflow execution logic 305 may automatically advance to the next workflow component. The “Patency Check” 510 workflow component may also include a verification step where a user confirms that no discernable indications of extravasation are present.

The “Connect & Purge Tubing” 509 workflow component shown in FIG. 5 may be a step performed by both the user and the medical fluid delivery system 300. The user may connect appropriate tubing to the injection device 301 and place the injection device in a proper orientation for purging. While the user is performing these steps, the user interface 302 may display textual and/or graphical instruction related to tubing attachment to aid the user. Once the tubing is connected and the injection device 301 is in the proper orientation, the user may indicate to the workflow execution logic 305 to proceed with the purging operation. The medical fluid delivery system 300 may then automatically perform a purging operation. The user could also manually control the purging operation.

Returning to FIG. 6, alternatively, the textual instruction 604 may reference a specific syringe size and/or medical fluid type in workflows where the administrator selected a default value for the “Load Prefill” 508 workflow component during workflow construction or where the protocol selected in the “Protocol Selection” 507 workflow component requires a specific size/type of contrast. For example, in place of the textual instruction 604 shown in FIG. 6, the textual instruction may read “Load 125 ml prefilled syringe onto injection device.” In another example, the type of contrast or brand name of the contrast may be included in the textual instruction 604.

The “Exit Workflow” button 607 may be used by the user to exit the current workflow and access additional functionality of the medical fluid delivery system 300. The medical fluid delivery system 300 may retain information related to the workflow such that the workflow may be resumed. For example, a user may exit the current workflow, perform a task and/or access a capability of the medical fluid delivery system 300 independent of the workflow, and then return to and continue the workflow. In this regard, the user may temporarily suspend the performance of the workflow. The “Exit Workflow” button 607 may also be used to abort and/or cancel the current workflow. After activating the “Exit Workflow” button 607, the medical fluid delivery system 300 may query the user as whether the user would like to save the current progress of the workflow for later resumption or permanently leave the workflow.

The user interface 302 outputs generated by the workflow execution logic 305 related to each workflow component may be simplified (relative to the user interface 302 outputs of the medical fluid delivery system 300 when not executing a workflow) to substantially show information and features related to the currently active workflow component. In this regard, the workflow may guide the user through the injection steps in a simplified manner, sequentially displaying instructions, where for each workflow component or portion thereof, substantially only what is needed for the user to complete that workflow component or portion thereof is displayed. Accordingly, each output related to a particular workflow component may contain little or no information about other workflow components. Such a system may reduce the amount of memorization required from a user and/or reduce the need for the user to rely on checklists or written instructions. This may consequently reduce training requirements for users of the workflow execution logic 305.

FIGS. 5 and 6 have been illustrated and described generally as touch screens. Other interfaces may be used by the medical fluid delivery system 300. For example, the user interface 302 may incorporate multifunction buttons along the perimeter of the screen, a keyboard, a keypad, a mouse, knobs, or any other appropriate input device or combination of input devices.

In an arrangement, workflows may be associated with particular injection protocols. In such an arrangement, a user may select a protocol to be run and the workflow execution logic 305 may then initiate a workflow associated with the protocol. In another arrangement, injection protocols may be associated with particular workflows. In such an arrangement, a user may select a workflow to be run and the workflow execution logic 305 may, at the appropriate time (e.g., during “Main Injection” 511 workflow component), initiate a protocol associated with the protocol. In still another arrangement (e.g., the arrangement being constructed in FIG. 5), the user may load a workflow and a step of the workflow may include protocol selection (e.g., Protocol Selection” 507 workflow component).

The workflow construction logic, workflow execution logic, power injector control logic, injection protocol setup logic, and patency check setup logic each may be implemented in any appropriate manner, including without limitation in any appropriate software, firmware, or hardware, using one or more platforms, using one or more processors, using memory of any appropriate type, using any single computer of any appropriate type or a multiple computers of any appropriate type and interconnected in any appropriate manner, or any combination thereof. Furthermore, each logic may be implemented at any single location or at multiple locations that are interconnected in any appropriate manner (e.g., via any type of network).

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain best modes known of practicing the invention and to enable others skilled in the art to utilize the invention in such, or other embodiments and with various modifications required by the particular application(s) or use(s) of the present invention. It is intended that the appended claims be construed to include alternative embodiments to the extent permitted by the prior art.

1. A medical fluid delivery system comprising:
an injection device;
a user interface; and
workflow construction logic operable to present a plurality of available workflow components to a user through said user interface, wherein said workflow construction logic is operable to receive a selection of a proper subset of said plurality of available workflow components from said user through said user interface, and wherein said workflow construction logic is operable to construct a workflow from said proper subset.

2. The medical fluid delivery system of claim 1, wherein said user interface is integral with said injection device.

3. The medical fluid delivery system of claim 2, wherein said injection device comprises said workflow construction logic.

4. The medical fluid delivery system of claim 1, wherein said plurality of available workflow components comprises mutually exclusive workflow components, wherein said workflow construction logic is operable to prevent at least one of simultaneous selection of more than one of said mutually exclusive components and simultaneous inclusion of more than one of said mutually exclusive components in said workflow.

5. The medical fluid delivery system of claim 1, wherein said plurality of available workflow components comprises workflow components related to at least one of: syringe type selection, syringe quantity selection, injection protocol selection, syringe loading, syringe re-loading, syringe filling, tubing set connecting and purging, patency check, test injection, main injection, results display, results printing, and tubing and syringe removal.

6. The medical fluid delivery system of claim 1, wherein said workflow construction logic allows for alteration of visual elements displayed on said user interface in association with at least a portion of said plurality of available workflow components.

7. The medical fluid delivery system of claim 6, wherein said visual elements comprise textual elements.

8. The medical fluid delivery system of claim 1, further comprising workflow execution logic operable to present said workflow to a user.

9. The medical fluid delivery system of claim 8, wherein said workflow execution logic generates at least one output on a display for each workflow component of said workflow.

10. The medical fluid delivery system of claim 8, wherein said workflow execution logic generates a sequence of outputs on a display in accordance with said workflow.

11. The medical fluid delivery system of claim 8, wherein said workflow execution logic comprises a suspension function that operates to temporarily suspend performance of said workflow, wherein while said workflow is temporarily suspended, said medical fluid delivery system is operable to perform functions that are not part of said workflow, wherein said workflow execution logic resumes after completion of said suspension function.

12. The medical fluid delivery system of claim 8, wherein said workflow execution logic operates to sequentially execute each workflow component of said proper subset of said plurality of available workflow components in a predetermined sequence.

13. The medical fluid delivery system of claim 1, further comprising a plurality of stored workflows.

14. The medical fluid delivery system of claim 13, further comprising a data input device that allows for selection of a workflow from said plurality of stored workflows.

15. A method of operating a medical fluid delivery system, said method comprising:
providing a medical fluid delivery system comprising a plurality of available workflow components; and
selecting a proper subset of workflow components from said plurality of available workflow components for inclusion within a workflow.

16. The method of claim 15, further comprising arranging workflow components of said proper subset to form said workflow.

17. The method of claim 16, wherein at least one of said selecting and arranging steps comprises preventing, by said medical fluid delivery system, inclusion within said workflow of a set of mutually exclusive workflow components.

18. The method of claim 17, further comprising displaying visual elements representing each workflow component of said set of mutually exclusive workflow components with at least one of: a common color, a common pattern, and a common shape.

19. The method of claim 16, wherein said arranging step comprises said medical fluid delivery system indicating to a user that a workflow component is in an inappropriate position relative to another workflow component.

20. The method of claim 15, further comprising displaying a plurality of visual elements on a user interface, wherein each of said plurality of visual elements represents at least one workflow component of said plurality of available workflow components.

21. The method of claim 20, wherein said selecting step comprises moving one of said visual elements from a first portion of said user interface to a second portion of said user interface.

22. The method of claim 21, wherein said first portion of said user interface comprises a plurality of visual elements representing unselected workflow components and said second portion of said user interface comprises a plurality of visual elements representing selected workflow components.

23. The method of claim 15, wherein said selecting step comprises including within said workflow a workflow component comprising an adjustable parameter.

24. The method of claim 23, further comprising inputting a default value for said adjustable parameter during at least one of said selecting and arranging steps.

25. The method of claim 23, further comprising inputting a default range for said adjustable parameter during at least one of said selecting and arranging steps.

26. The method of claim 24, further comprising inputting a value for said adjustable parameter after completion of said selecting and arranging steps.

27. The method of claim 15, further comprising displaying a representation of a partially constructed workflow during said selecting and arranging steps.

28. The method of claim 15, further comprising customizing textual elements of a display associated with a workflow component of said proper subset.

29. The method of claim 15, further comprising performing at least a portion of said workflow to prepare said medical fluid delivery system for injecting medical fluid into a patient.

30. The method of claim 29, wherein said at least a portion of said workflow comprises workflow components related to mounting a syringe onto said medical fluid delivery system.

31. The method of claim 29, wherein said at least a portion of said workflow comprises workflow components related to purging portions of said medical fluid delivery system of air.

32. The method of claim 29, wherein said performing at least a portion of said workflow includes at least one of displaying instructions for a clinician and automatic performance of at least a portion of a workflow component of said proper subset by said medical fluid delivery system.

33. The method of claim 29, wherein said performing step comprises sequentially displaying a first display associated with a first workflow component and a second display associated with a second workflow component, wherein said first display is free of information related to said second workflow component and said second display is free of information related to said first workflow component.

34. The method of claim 15, further comprising discharging medical fluid from said medical fluid delivery system.

35. The method of claim 15, further comprising executing said workflow to inject medical fluid into a patient.

36. The method of claim 35, wherein said executing step comprises selecting a protocol, wherein said medical fluid delivery system selects said workflow based on said protocol selected in said selecting a protocol step.

37. The method of claim 35, wherein said selecting step is performed by a medical fluid delivery system administrator, wherein said executing step is initiated by a clinician.

38. The method of claim 35, wherein said selecting step comprises assigning a default value related to a workflow component of said proper subset, wherein said executing step is performed using said default value.

39. The method of claim 38, further comprising overriding, during said executing step, said default value.

40. The method of claim 38, wherein said assigning a default value is performed by a user.

41. The method of claim 35, wherein said selecting step comprises assigning a default range related to a workflow component of said proper subset, wherein said executing step is performed using said default range.

42. The method of claim 41, further comprising generating a warning during said executing step indicating that a value related to said workflow component is outside of said range.

43. The method of claim 41, wherein said assigning a default range is performed by a user.

44. The method of claim 35, wherein said executing step comprises sequentially performing said proper subset of workflow components.

45. The method of claim 35, wherein said executing step comprises at least one of presenting instructions for a clinician on a first display and automatic performance of at least a portion of a workflow component of said proper subset by said medical fluid delivery system.

46. The method of claim 35, wherein said executing step comprises sequentially presenting a first output on a first display associated with a first workflow component and presenting a second output on said first display associated with a second workflow component, wherein said first output is free of information related to said second workflow component and said second output is free of information related to said first workflow component.

47. The method of claim 46, wherein presenting said second output associated with said second workflow component occurs automatically after completion of said first workflow component.

48. The method of claim 35, further comprising:
exiting said workflow prior to completion of each workflow component; and
performing a task with said medical fluid delivery system that deviates from said workflow.

49. The method of claim 15, further comprising providing a password to said medical fluid delivery system to enable performance of said selecting step.

50. The method of claim 15, further comprising saving said workflow after said selecting step.

51. The method of claim 50, further comprising selecting said workflow after said saving step and before said executing step.

52. The method of claim 51, wherein said selecting step comprises choosing said workflow from a plurality of stored workflows.

53. The method of claim 52, wherein said choosing step is performed with a data input device.

54. The method of claim 50, further comprising designating said workflow as a default workflow after said saving step and before said executing step.

55. A medical fluid delivery system comprising an injection device and a memory unit, said memory unit comprising:
at least one injection protocol containing injection execution parameters;
a plurality of workflow components; and
a first workflow comprising a first proper subset of said plurality of workflow components, wherein said first workflow does not include any workflow components not contained in said first proper subset.

56. The medical fluid delivery system of claim 55, further comprising a plurality of injection protocols each containing injection execution parameters.

57. The medical fluid delivery system of claim 55, further comprising a second workflow comprising a second proper subset of said plurality of workflow components, wherein said second workflow does not include any workflow components not contained in said second proper subset, wherein said first proper subset is different than said second proper subset.
